(19) United States
(12) Patent Application Publication
(10) Pub. No.: US 2023/0033398 A1
(43) Pub. Date: Feb. 2, 2023

(54) FULL-DUPLEX EPG SYSTEM AND ELECTRO-OPTICAL PERCUTANEOUS LEAD

(71) Applicant: Wavegate Corporation, Lake Charles, LA (US)

(72) Inventors: Dennis Patrick O'Neal, Coppell, TX (US); Erich W. Wolf, II, Lake Charles, LA (US)

(73) Assignee: Wavegate Corporation, Lake Charles, LA (US)

(21) Appl. No.: 17/815,494

(22) Filed: Jul. 27, 2022

Related U.S. Application Data
(63) Continuation-in-part of application No. 17/815,482, filed on Jul. 27, 2022.
(60) Provisional application No. 63/203,649, filed on Jul. 27, 2021.

(51) Int. Cl. A61N 1/36; A61N 1/05

(52) U.S. Cl. CPC A61N 1/36062; A61N 1/0551; A61N 1/36071; A61N 1/36132; A61N 1/36135; A61N 1/36157

(57) ABSTRACT

The invention provides an EPG system and lead configuration which boasts both a novel optical folding assembly and compact package size. The percutaneous leads provided offer additional advantages over the prior art including integral formation of optical and electrical components in a compact size.

### CROSS-REFERENCE TO RELATED APPLICATIONS

This application is a continuation-in-part of U.S. application Ser. No. 17/815,482 filed on Jul. 27, 2022. This application also claims priority benefit from U.S. Provisional Application No. 63/203,649 filed on Jul. 27, 2021. The patent applications identified above are incorporated here by reference in its entirety to provide continuity of disclosure.

### FIELD OF THE INVENTION

The present invention relates to external pulse generator (“EPG”) systems for spinal cord stimulation (“SCS”).

### BACKGROUND OF THE INVENTION

Chronic pain may arise from a variety of conditions, most notably from nerve injury as in the case of neuropathic pain, or from chronic stimulation of mechanical nociceptors such as with spinal pain. Functional ability may be severely impacted by pain, which often is refractory to pharmacological and surgical treatment. In such cases, SCS can be an effective treatment for pain by modulating physiological transmission of pain signals from the periphery to the brain. This may be achieved by applying electrical impulses to the spinal cord via an electrode array implanted adjacent the spinal canal.

Referring to FIGS. 1, 2 and 3, a typical EPG system of the prior art will be described. Spinal column 1 is shown to have a number of vertebrae, categorized into four sections or types: lumbar vertebrae 2, thoracic vertebrae 3, cervical vertebrae 4 and sacral vertebrae 5. Cervical vertebrae 4 include the 1st cervical vertebra (C1) through the 7th cervical vertebra (C7). Just below the 7th cervical vertebra is the first of twelve thoracic vertebrae 3 including the 1st thoracic vertebra (T1) through the 12th thoracic vertebra (T12). Just below the 12th thoracic vertebrae 3, are five lumbar vertebrae 2 including the 1st lumbar vertebra (L1) through the 5th lumbar vertebra (L5), the 5th lumbar vertebra being attached to sacral vertebrae 5 (S1 to S5), sacral vertebrae 5 being naturally fused together in the adult.

Representative vertebra 10, a thoracic vertebra, is shown to have a number of notable features which are in general shared with lumbar vertebrae 2 and cervical vertebrae 4. The thick oval segment of bone forming the anterior aspect of vertebra 10 is vertebral body 12. Vertebral body 12 is attached to bony vertebral arch 13 through which spinal nerves 11 run. Vertebral arch 13, forming the posterior of vertebra 10, is comprised of two pedicles 14, which are short stout processes that extend from the sides of vertebral body 12 and bilateral laminae 15. The broad flat plates that project from pedicles 14 join in a triangle to form a hollow archway, spinal canal 16. Spinous process 17 protrudes from the junction of bilateral laminae 15. Transverse processes 18 project from the junction of pedicles 14 and bilateral laminae 15. The structures of the vertebral arch protect spinal cord 20 and spinal nerves 11 that run through the spinal canal.

Surrounding spinal cord 20 is dura 21 that contains cerebrospinal fluid (CSF) 22. Epidural space 24 is the space within the spinal canal lying outside the dura.

One or more electrodes 30 are positioned in epidural space 24 between dura 21 and the walls of spinal canal 16 towards the dorsal aspect of the spinal canal nearest bilateral laminae 15 and spinous process 17. Electrode 30 has electrode leads 31 which are connected to EPG 32 and controller 33.

EPG 32 provides the electrical stimulation in the form of current pulses to the spinal cord through lead 31 to electrode 30. The pulses generate an electric field. The electric field impinges on targeted neurons of the spinal cord and disrupts the perception of pain. The amplitude of the electrical field is critical to success of spinal cord stimulation. An inadequate electric field will fail to depolarize the targeted neurons, rendering the treatment ineffective. An excess electric field stimulates neighboring cell populations which results in a noxious stimulation.

Establishing a consistent, therapeutic, and non-noxious level of stimulation is predicated upon establishing an ideal current density within the spinal cord's targeted neurons. Fundamentally, this should be a simple matter of establishing an optimal electrode current given the local bulk conductivity of the surrounding tissues. But in practice, the optimal electrode current changes as a function of patient position and activity due to motion of the spinal cord as the spinal cord floats in cerebrospinal fluid within the spinal canal. Significant changes in distance between the epidural electrode array and the targeted spinal cord neurons have been shown to occur. Consequently, optimal stimulation requires dynamic adjustment of the electrode stimulating current as a function of distance between the electrode array and the spinal cord.

Dynamic modulation of spinal cord stimulator electrode current as a function of distance between the electrode array and the spinal cord thus has several benefits. Excess stimulation current can be avoided, thus reducing the prospects of noxious stimulation and potentially reducing device power consumption. Inadequate stimulation current can also be avoided, thus eliminating periods of compromised therapeutic efficacy.

Dynamic modulation of electrode current can be controlled through the use of optical reflectometry to determine the thickness of the dorsal cerebrospinal fluid (dCSF) column between the spinal cord and the electrode array. An optical signal is transmitted into the surrounding tissue and collected by a sensor to calculate the approximate distance between the electrode and the spinal cord. The stimulus magnitude is modified accordingly to provide the optimal current for pain relief. Examples of this technology are shown in U.S. Pat. Nos. 10,035,019 and 9,656,097, both to Wolf II, and both incorporated herein by reference.

One challenge to EPG systems is proper interpretation of the reflected optical signal. Half duplex systems of the prior art require that multiple surgical leads be precisely placed by the targeted neurons. Such placement during surgery is difficult. The problem is further exacerbated by lead migration, which may disalign the optical feedback and the targeted neurons.

Yet another challenge to EPG systems is power constraints and heat generation. Power usage of the EPG must be kept to a minimum in order to assure long term battery life of the EPG package. Therefore, power consumption must be as low as possible.

Another challenge to EPG systems is initial lead alignment during implantation. Stylets must be present in the leads while the stimulation signal is active so that the position of the electrodes may be adjusted using patient feedback. However, the stylets must be removed for connection to the optical feedback system. Hence, difficult and time-consuming removal and reinsertion of the stylets in the lead lumens is often required to accurately position the leads.

The prior art has attempted to address these challenges in a number of ways, yet all have fallen short.

For example, U.S. Pat. No. 9,656,097 to Wolf, II describe a full duplex implanted pulse generator (“IPG”) lead, which allows both a transmit ray and receive ray to travel down the same fiber. However, Wolf discloses use of a circulator to separate the two rays. A circulator is not practical in a small EPG package and because the internal signal losses between optical components would be prohibitive.

As another example, U.S. Pat. No. 7,742,817 to Malinowski, et al. describes an IPG with connectors for electrical leads and an epoxy coating for biocompatibility. However, Malinowski does not disclose the use of optical feedback to achieve proper pulse strength.

As another example, U.S. Publication No. 2021/0001114 to Wolf, II, discloses coupling of an optical lead to an IPG header adjacent a ruby passthrough window, vertically aligned with a photo detector. However, Wolf fails to disclose a way to optimize fiber coupling in a compact package size.

U.S. Publication No. 2018/0154152 to Chabrol discloses a system for deep brain stimulation using a probe with stimulation electrodes and a light emitting optical fiber. However, Chabrol fails to address using the light signal to control a stimulation signal to the spinal cord. Chabrol also fails to address optical coupling in a full-duplex optical system.

Deficiencies exist in the prior art related to optical coupling of the fibers to the internal components of the EPG package and optical signal separation. Thus, there is a need in the art for an improved EPG including optimal signal separation, leads and electrodes which provide a stable optical signal while optimizing fiber coupling.

### BRIEF DESCRIPTION OF THE DRAWINGS

In the detailed description of the preferred embodiments presented below, reference is made to the accompanying drawings.

FIG. 1 is a side view of the human spine showing the approximate position of a percutaneous lead and EPG for spinal cord stimulation.

FIG. 2 shows an axial view of a thoracic vertebra indicating the position of the spinal cord and a percutaneous lead pair.

FIG. 3 shows a sagittal cross-sectional view of the human spine showing the approximate position of a percutaneous lead.

FIG. 4 shows a schematic diagram of an EPG system of a preferred embodiment.

FIG. 5 is an isometric view of a preferred EPG device.

FIG. 6 is an exploded isometric view of a preferred EPG device.

FIG. 7 is an exploded isometric view of a preferred EPG case.

FIG. 8 is an exploded isometric view of a preferred EPG header.

FIG. 9 is an exploded isometric view of a preferred die stack.

FIG. 10A is a top view of a preferred redirector.

FIG. 10B is a side view of a preferred redirector.

FIG. 10C is a side view of a preferred redirector.

FIG. 10D is an end view of a preferred redirector.

FIG. 10E is an isometric view of a preferred redirector.

FIG. 11 is a partial cross-section view of a preferred header assembly.

FIG. 12 is an isometric view of a lead assembly of a preferred embodiment.

FIG. 13 is a cross-section view of a lead assembly of a preferred embodiment.

FIG. 14 is a flowchart of a preferred control program for operation of the EPG.

FIG. 15 is a flowchart of a preferred method of use of the EPG.

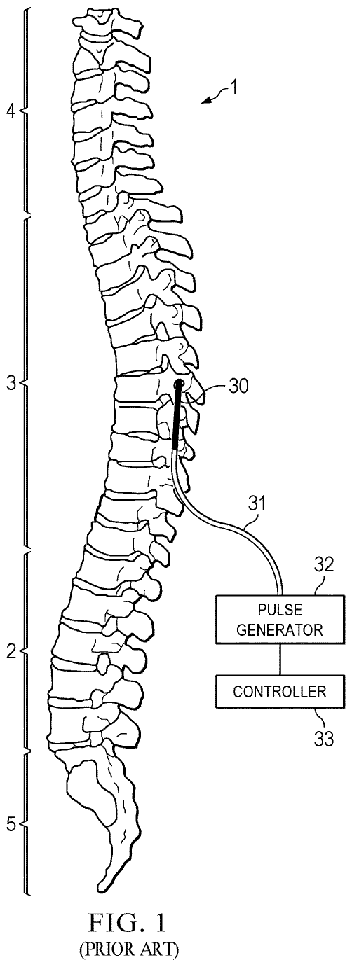

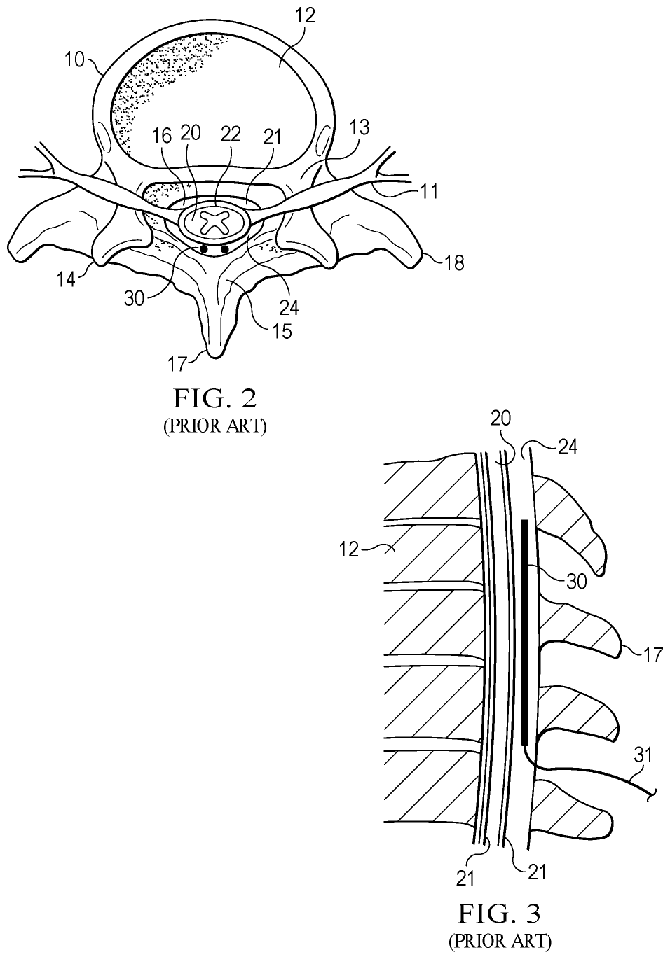

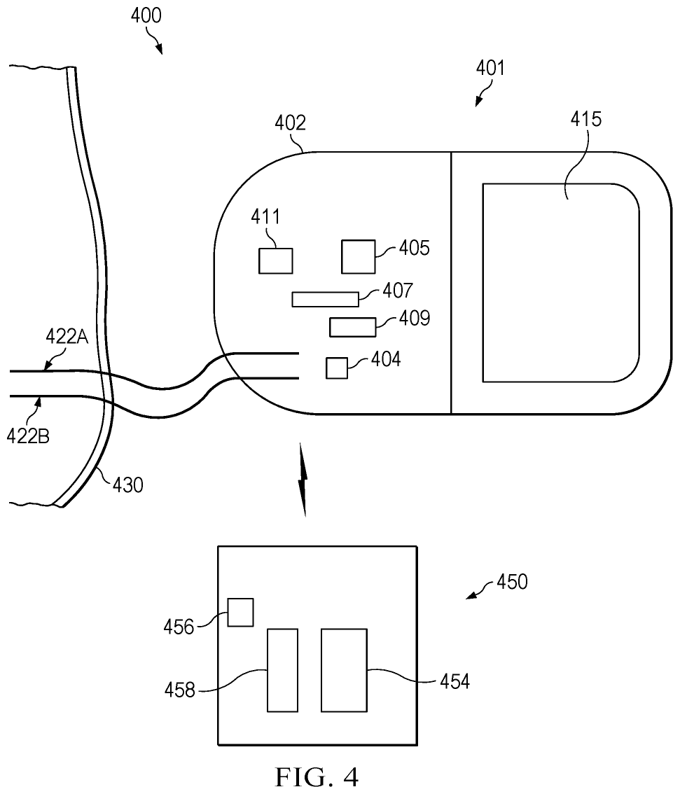

### DETAILED DESCRIPTION OF THE INVENTION

In the description that follows, like parts are marked throughout the specification and figures for the same numerals. The figures are not necessarily drawn to scale and may be shown in exaggerated or generalized form in the interest of clarity and conciseness. Unless otherwise noted, all tolerances and uses of the term “about” indicate plus or minus 5%.

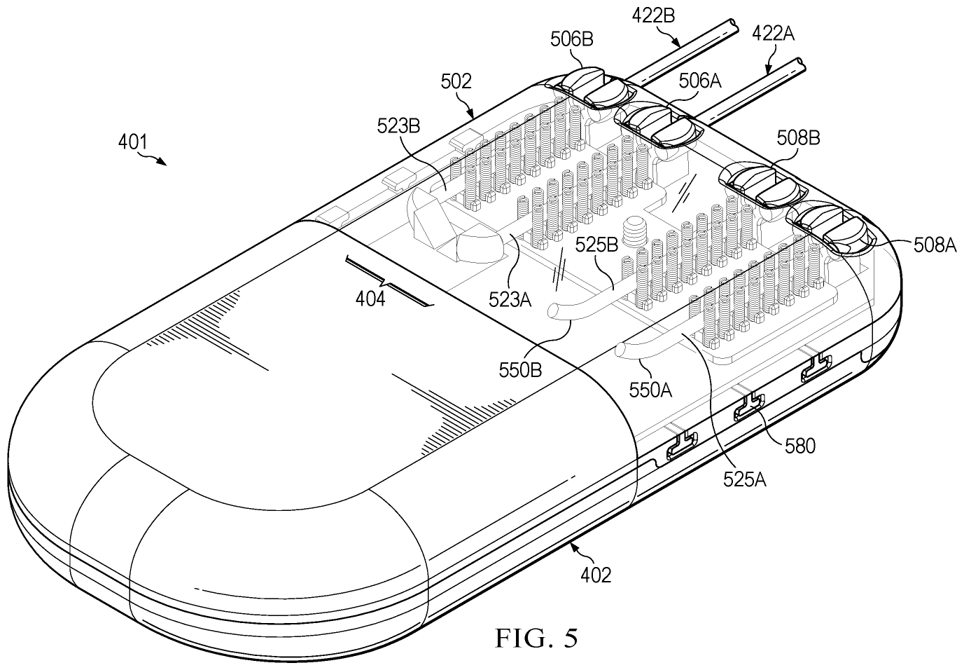

Referring then to FIG. 4, preferred embodiment of stimulation system 400 will be further described. Stimulation system 400 further comprises EPG 401 in operative communication with controller 450.

EPG 401 is housed in case 402, as will be further described. Case 402 houses the operative components of the EPG and serves to anchor leads 422A and 422B, as will be further described. The leads extend from the EPG, through dermis 430 terminating at the spinal cord.

The operative components of the system comprise optical folding assembly 404, optically aligned with leads 422A and 422B. Optical folding assembly 404 directs optical signals from leads 422A and 422B to optical signal processor 405. Optical signal processor 405 is operatively connected to main processor 407, which controls the functions of the EPG, as will be further described. Main processor 407 is operatively connected to signal generator 409, which generates electrical stimulation signals which are transmitted through leads 422A and 422B to the spinal cord to targeted nerve populations, as will be further described. Communications circuit 411 is also operatively connected to main processor 407. Main processor 407 receives programming instructions and control signals from the communications circuit, as will be further described.

EPG 401 includes battery 415. Battery 415 is operatively connected to all the electrical components of the system.

Controller 450 includes main processor 454, which provides the functions of the controller. Main processor 454 is connected to I/O keyboard and display unit 458, which is fixed in an exterior casing. Main processor 454 is operatively connected to communications circuit 456. Communications circuit 456 is wirelessly connected to communications circuit 411. Main processor 454 includes sufficient memory to receive instructions from I/O keyboard and display unit 458 and transfer them through communications circuit 456 and communications circuit 411 to main processor 407 for controlling the operation of EPG 401.

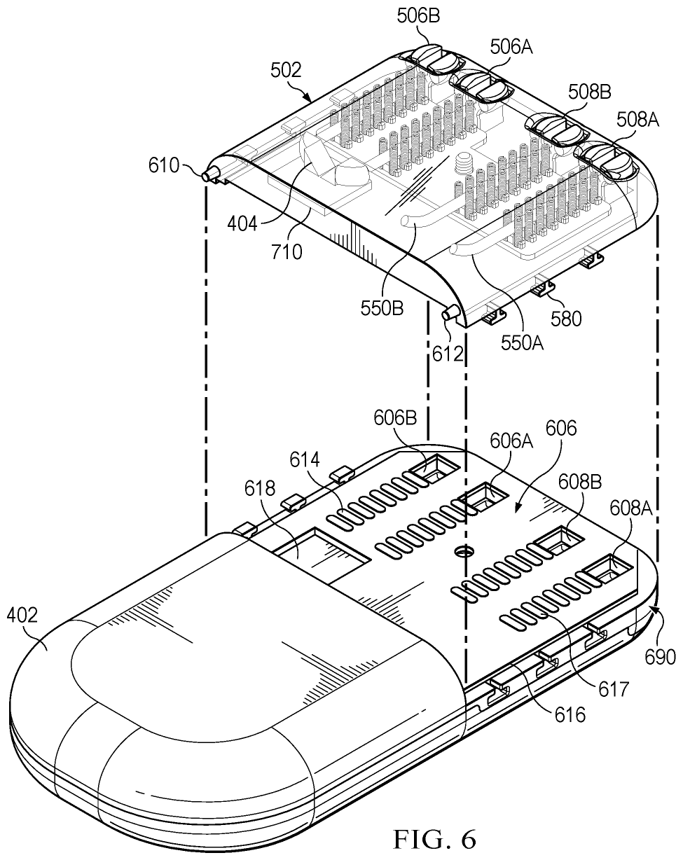

Referring then to FIG. 5, EPG 401 will be further described.

Case 402 mechanically supports header assembly 502 by a plurality of mechanical connectors 580. The header assembly preferably is manufactured from a transparent epoxy resin which serves to fix the optical and electrical components in place.

Header assembly 502 includes optical folding assembly 404. Optical folding assembly 404 is optically aligned with lead retainer holes 523A and 523B. Leads 422A and 422B are removably positioned in and optically aligned by lead retainer holes 523A and 523B. Leads 422A and 422B are held in place in the lead retainer holes by virtue of retainer clips 506A and 506B, respectively. The leads abut optical folding assembly 404, as will be further described. Leads 422A and 422B are also alternately and removably positioned in lead retainer holes 525A and 525B and held in place by clips 508A and 508B. Lead retainer hole 525A terminates in open port 550A. Likewise, lead retainer hole 525B terminates in open port 550B. In use, the leads are positioned first in lead retainer holes 525A and 525B for testing during lead implantation and then moved to lead retainer holes 523A and 523B, during steady state operation of the EPG. During testing and lead adjustment, the stylet for lead 422B extends through port 550B and the stylet for lead 422A extends through port 550A, as will be further described.

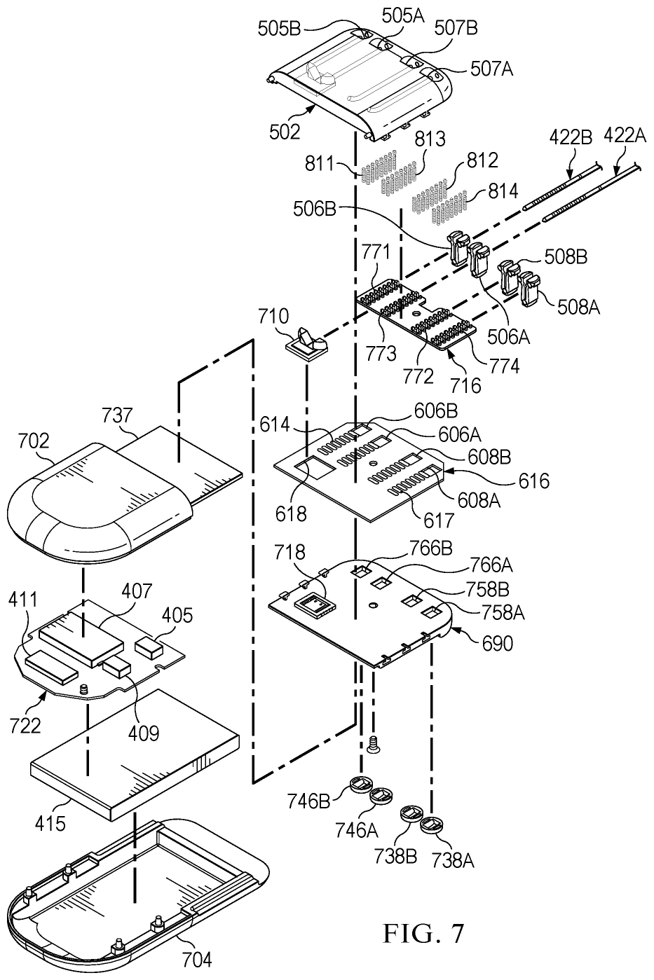

Referring then to FIG. 6, EPG 401 will be further described.

Case 402 further comprises header bay 606. The header bay is a generally rectangular indention in the case. Header bay 606 houses header assembly 502.

Connector card 616 is positioned at the base of header bay 606. Connector card 616 is rigidly fixed to chassis 690, as will be further described. Connector card 616 includes rectangular receiving window 618 and electrical pass-through holes 614. Likewise, connector card 616 includes electrical pass-through holes 617. The receiving windows and the electrical pass-through holes allow for connections between header assembly 502 and the internal components of the EPG.

Header assembly 502 includes integrally formed positioning pin 610 and positioning pin 612. The positioning pins mate with aligning holes in the case (not shown) to aid in optically aligning the header with the case.

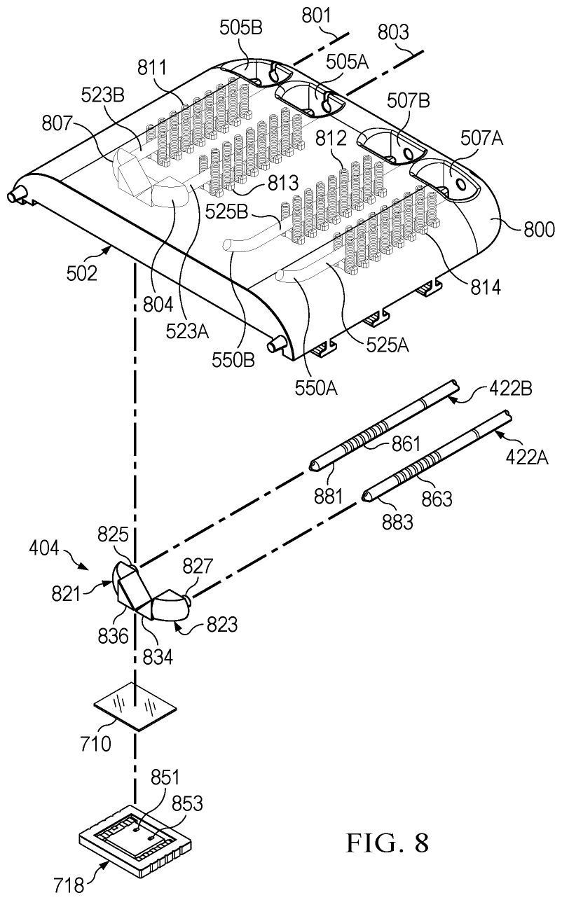

Referring to FIG. 7, the optical components of the EPG will be further described.

Case 402 includes top section 702 joined to bottom section 704. Top section 702 and bottom section 704 are preferably shells manufactured from a resilient thermoplastic.

Top section 702 is integrally formed with chassis support 737. Chassis support 737 is generally a rectangular shelf which fits beneath and nests within chassis 690. Chassis 690 further comprises clip receiver holes 766B, 766A, 758B and 758A. Clip receiver holes 766B, 766A, 758B and 758A, are positioned directly beneath clip receiver holes 606B, 606A, 608B and 608A, in connector card 616. Chassis 690 mechanically secures and positions die stack 718. Die stack 718 positioned directly beneath receiving window 618.

Optical window 710 is positioned within receiving window 618. The optical window is preferably welded and sealed to the connector card.

Contact positioning card 716 is rigidly fixed to connector card 616. Contact positioning card 716 includes connectors 771, 773, 772 and 774. Connectors 771 and 773 feed through electrical pass-through holes 614 and are electrically connected to the main processor. Connectors 772 and 774 feed through electrical pass-through holes 617 and are electrically connected to the main processor.

Spring contacts 811, 813, 812 and 814 are physically and electrically connected to connectors 771, 773, 772 and 774, respectively. Electrical connections between the spring contacts and the main processor allow for transmission of stimulation current signals to the leads, as will be further described.

Header assembly 502 further comprises vertical holes 505B, 505A, 507B and 507A. Retainer clips 506B, 506A, 508B and 508A fit into vertical holes 505B, 505A, 507B, 507A, through clip receiver holes 606B, 606A, 608B and 608A, and into clip receiver holes 766B, 766A, 758B and 758A, where they are secured by fasteners 746B, 746A, 738B and 738A, respectively. When positioned in the vertical holes, the retainer clips are compressed about the leads which are secured in place along their proper axes.

Case 402 houses processor card 722. Processor card 722 structurally and electrically connects main processor 407 to optical signal processor 405, signal generator 409 and communications circuit 411. Processor card 722 is also electrically connected to die stack 718 in order to communicate electrical signals to the die stack to activate the lasers and measure current from the photodiodes, as will be further described. Processor card 722 is also electrically connected to connector card 616 in order to transmit stimulation current signals to the leads, as will be further described.

Case 402 houses processor card 722. Processor card 722 structurally and electrically connects main processor 407 to optical signal processor 405, signal generator 409 and communications circuit 411. In a preferred embodiment, main processor 407 is Part No. MSP430, available from Texas Instruments of Dallas, Tex. Signal generator 409 is available under the tradename Saturn, available from Cactus Semiconductor. Communications circuit 411 is preferably Part No. ZL70103, available from Microsemi Corporation of Aliso Viejo, Calif. Optical signal processor 405 are both preferably Part No. ADPD4100, available from Analog Devices of Wilmington, Mass.

Case 402 further houses battery 415 which is electrically connected to processor card 722. The operational components of the EPG are preferably positioned adjacent held in place by an epoxy encapsulation. After encapsulation, bottom section 704 is sealed to top section 702 by mechanical connectors and a suitable industrial adhesive.

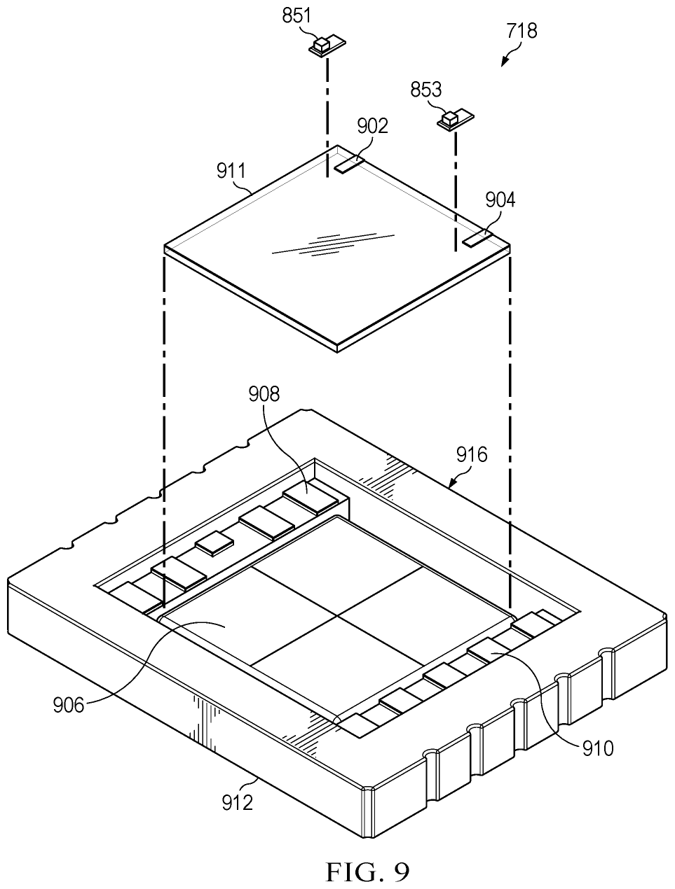

Referring then to FIG. 8, header assembly 502 will be further described.

Header assembly 502 includes header body 800. Header body 800 preferably is manufactured from a transparent epoxy resin, or acrylic plastic, cast and machined to tolerance. Header body 800 is formed to fit seamlessly within header bay 606.

Header body 800 includes lead retainer holes 523B, 523A, 525B and 525A. Lead retainer hole 523A has central optical axis 803. Lead retainer hold 523B has central optical axis 801. The optical axes, preferably, are generally parallel.

Lead retainer hole 525A includes metallic spring contacts 814. In a preferred embodiment, eight pairs of vertical spring contacts are included. Each opposing pair is individually addressable by the main processor so as to communicate stimulation signals to a single cylindrical lead contact, as will be further described. Lead retainer hole 525A intersects vertical hole 507A.

Lead retainer hole 525B includes metallic spring contacts 812. In a preferred embodiment, eight pairs of vertical spring contacts are included. Each pair is individually addressable by the main processor. Lead retainer hole 525B intersects vertical hole 507B.

Lead retainer hole 523A includes metallic spring contacts 813. In a preferred embodiment, metallic pairs of vertical spring contacts are included, each pair individually addressable by the main processor. Lead retainer hole 523A intersects vertical hole 505A. Lead retainer hole 523A terminates in cavity 804, as will be further described.

Lead retainer hole 523B includes metallic spring contacts 811. In a preferred embodiment, metallic pairs of vertical spring contacts are included, each pair individually addressable by the main processor. Lead retainer hole 523B intersects vertical hole 505B. Lead retainer hole 523B terminates in cavity 807, as will be further described.

Optical folding assembly 404 further comprises parabolic redirector 821 and parabolic redirector 823. The parabolic redirectors are diametrically opposed. Each serves to turn the optical axis of the fibers about 90 degrees horizontally inward and then about 90 degrees vertically downward toward the die stack. Parabolic redirector 821 includes integral lens 825 and prism 836. Parabolic redirector 823 includes integral lens 827 and prism 834. Parabolic redirector 821 is rigidly secured in cavity 807. Parabolic redirector 823 is rigidly secured in cavity 804. Integral lens 825 is optically aligned with optical axis 801. Integral lens 827 is optically aligned with optical axis 803.

Parabolic redirector 821 and parabolic redirector 823 are positioned by cavity 807 and cavity 804 in contact with and adjacent to optical window 710, as will be further described. In a preferred embodiment, the parabolic redirectors are cast in place in the header.

Parabolic redirector 821 will be referenced to as a “left hand” redirector. Parabolic redirector 823 will be referred to as a “right hand” redirector.

Die stack 718 is positioned directly below and in contact with optical window 710. Die stack 718 includes VCSEL 851 and VCSEL 853, as will be further described. VCSEL 851 is positioned generally perpendicular to and beneath prism 836. Likewise, VCSEL 853 is positioned generally perpendicular to and centered beneath prism 834.

In each case, the VCSELs are capable of emitting light in an intrinsic wavelength range, but preferably in the range of about 400-810 nanometers or from blue (about 400 nanometers to about 500 nanometers), to green (about 520 nanometers to about 532 nanometers) to near IR (about 700 nanometers to about 810 nanometers). In a preferred embodiment, each VCSEL is Part No. V00146, available from Vixar, Inc. of Plymouth, Minn. Each laser produces about 10 milliwatts in the range of near IR and about 5 milliwatts in the blue range. Other lasers may be utilized in the wavelength range of about 400-580 nanometers (blue, aqua, green and yellow) as well as other visible ranges.

Spring contacts 812 are designed to engage and electrically contact rings 861 of lead 422B, as will be further described. Spring contacts 814 are designed to engage and electrically contact rings 863 of lead 422A, as will be further described.

Spring contacts 811 are designed to engage in electrically contact rings 861 of lead 422B, as will be further described. Spring contacts 813 are designed to engage and electrically contact rings 863 of lead 422A, as will be further described.

When lead 422B is positioned in lead retainer hole 523B, it is aligned with optical axis 801. When lead 422A is positioned in lead retainer hole 523A, it is aligned with optical axis 803.

Lead 422B terminates in collet 881, as will be further described. Lead 422A terminates in collet 883, as will be further described. When positioned in lead retainer hole 523B, lead 422B and collet 881 are held adjacent to integral lens 825 by retainer clip 506B. When positioned in lead retainer hole 523A, lead 422A and collet 883 are held adjacent integral lens 827 by retainer clip 506A. In all cases, an index matching gel is provided to minimize Fresnel reflections between the lenses and the leads.

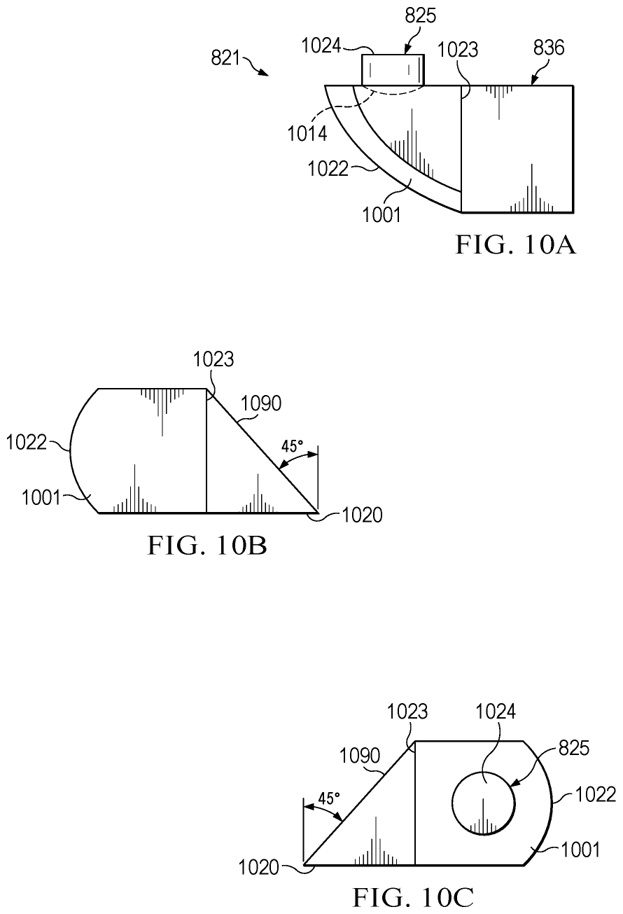

Referring in to FIG. 9, die stack 718 will be further described.

Die stack 718 includes photodiode 916. In a preferred embodiment, photodiode 916 is Part No. S5980-09(ESI), available from Hamamatsu Photonics K.K. of Shizuoka, Japan. Photodiode 914 further comprises housing 912. Housing 912 is preferably a ceramic composite. Housing 912 is held in position on the chassis by epoxy and rigidly fixes the position of photoreceiver 906. Photoreceiver 906 typically provides a sensitivity of about 0.72 A/W. Photoreceiver 906 is bounded by electrical contacts 908 and 910 which operatively connect the photodiode to the optical signal processor by traces on the chassis connected to the processor card (not shown). Cover plate 911 is positioned adjacent to and in contact with photoreceiver 906. In a preferred embodiment, cover plate 911 is formed of a crystal glass ground and polished to have optically parallel opposing faces. Cover plate 911 includes gold trace 902 and gold trace 904. The gold traces are preferably deposited on the glass using photolithography or vapor deposition. VCSEL 851 is rigidly fixed to cover plate 911 adjacent to and in electrical contact with gold trace 902. VCSEL 853 is rigidly fixed to cover plate 911 adjacent to and in electrical contact with gold trace 904. The gold traces provide power to the VCSELs and allow the controller to select which wavelength laser to activate, as will be further described. Gold trace 902, gold trace 904, and contacts 908 and 910 are electrically connected to optical signal processor 405 and main processor 407 by traces on the chassis to the processor card (not shown).

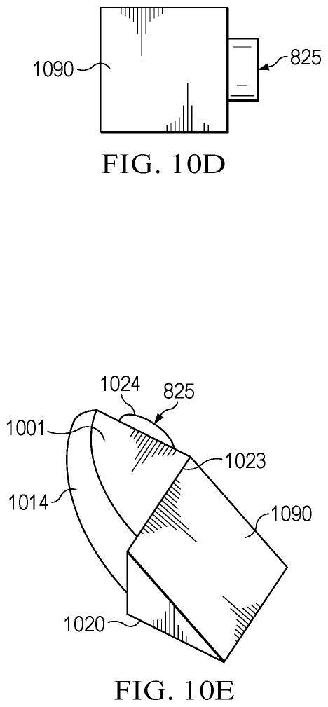

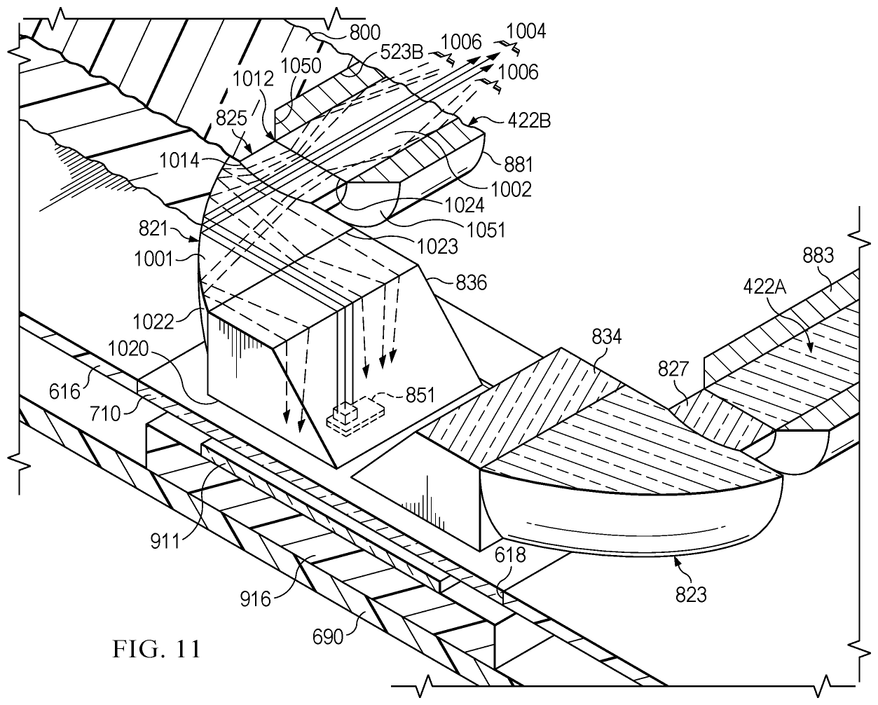

Referring then to FIGS. 10A, 10B, 10C, 10D, 10E and 11, parabolic redirector 821, will be further described. It should be understood that both parabolic redirectors are functionally identical and structurally similar, with the exception of being right or left handed. Hence, only one will be described here as an example.

Parabolic redirector 821 is further comprised of parabolic body 1001, integral lens 825 and prism 836. Parabolic body 1001, integral lens 825 and prism 836 are preferably integrally formed from a crystal glass having an index of refraction of between about 1.46 and 1.68.

Parabolic body 1001 includes parabolic surface 1022. Parabolic surface 1022 is preferably a paraboloid having a curvature designed to produce one focal point at the center of interface surface 1023 and another focal point at convex surface 1014 and aligned to the optical axis of lead 422B.

Parabolic surface 1022 is preferably polished. Parabolic surface 1022 preferably includes an exterior reflective coating of vapor deposited silver. In another embodiment, parabolic surface 1022 may be coated with a titanium dioxide compound.

Prism 836 further includes interface surface 1020. Interface surface 1020 is flat to within an acceptable optical tolerance. Preferably, an index matching material, such as an epoxy is resident between the interface surface and the optical window to minimize signal loss.

Parabolic redirector 821 is further comprised of integral lens 825. Integral lens 825 is a collimating optical element which includes convex lens surface 1014 directed inward toward parabolic surface 1022. In one preferred embodiment, convex lens surface 1014 is bonded to parabolic body 1001 with an index matching epoxy. In another preferred embodiment, the parabolic body and the convex lens are integrally formed. In this case, the convex lens surface is created by an appropriate density change between integral lens 825 and parabolic body 1001. Integral lens 825 includes interface surface 1024. Interface surface 1024 is preferably ground flat within appropriate optical tolerances.

Parabolic body 1001 is fixed to prism 836 at interface surface 1023. Interface surface 1023 is preferably an index matching epoxy. In another preferred embodiment, parabolic body 1001 and prism 836 are integrally formed.

Prism 836 includes angled surface 1090 and interface surface 1020. Angled surface 1090 is polished flat, and forms about a 45 degree angle with the vertical plane and interface surface 1020. Angled surface 1090 is preferably polished and includes a highly reflective coating such as vapor deposited silicon or titanium dioxide.

Interface surface 1020 is positioned adjacent optical window 710. Interface surface 1020 may be fixed to optical window 710 with an index matching epoxy. In another preferred embodiment, interface surface 1020 may be positioned adjacent optical window 710 with an index matching gel and fixed in place with a suitable epoxy adhesive.

Header body 800 includes frustroconical receiver surface 1050 at the proximal end of lead retainer hole 523B. Likewise, collet 881 includes frustroconical surface 1051. Interface surface 1024 is positioned parallel to and against fiber 1002 of lead 422B at optical interface 1012 and held in place by the interference between frustroconical surface 1051 and frustroconical receiver surface 1050 of lead retainer hole 523B.

Optical window 710 is positioned within receiving window 618. The interface between receiving window 618 and connector card 616 fixes the vertical optical axis of VCSEL 851 toward angled surface 1090 of prism 836, where transmit rays 1004 from the VCSEL are reflected about 90 degrees from the vertical to the horizontal and directed toward parabolic surface 1022.

The parabolic surface reflects the transmit rays about 90 degrees clockwise horizontally and then aligns them through integral lens 825 which collimates them and directs them into fiber 1002, along optical axis 801, where they exit the fiber at its distal end toward the spinal cord. Upon reflecting from the spinal cord, receive rays 1006 are collected and retransmitted through fiber 1002 back to the parabolic redirector. Receive rays 1006 are not well aligned along the fiber and so impact integral lens 825 at various angles. Receive rays 1006 are expanded by convex lens surface 1014 where they are incident upon parabolic surface 1022. Parabolic surface 1022 collects the receive rays and reflects them about 90 degrees counterclockwise horizontally toward angled surface 1090 of prism 836. Prism 836 reflects the receive rays vertically downward where they are incident on optical window 710. Receive rays 1006 pass through optical window 710 and are incident on cover plate 911 where they are directed toward photodiode 916 surrounding the VCSEL.

Photodiode 916 converts receive rays 1006 into electrical signals which are communicated to the optical signal processor 405 for processing, as will be further described.

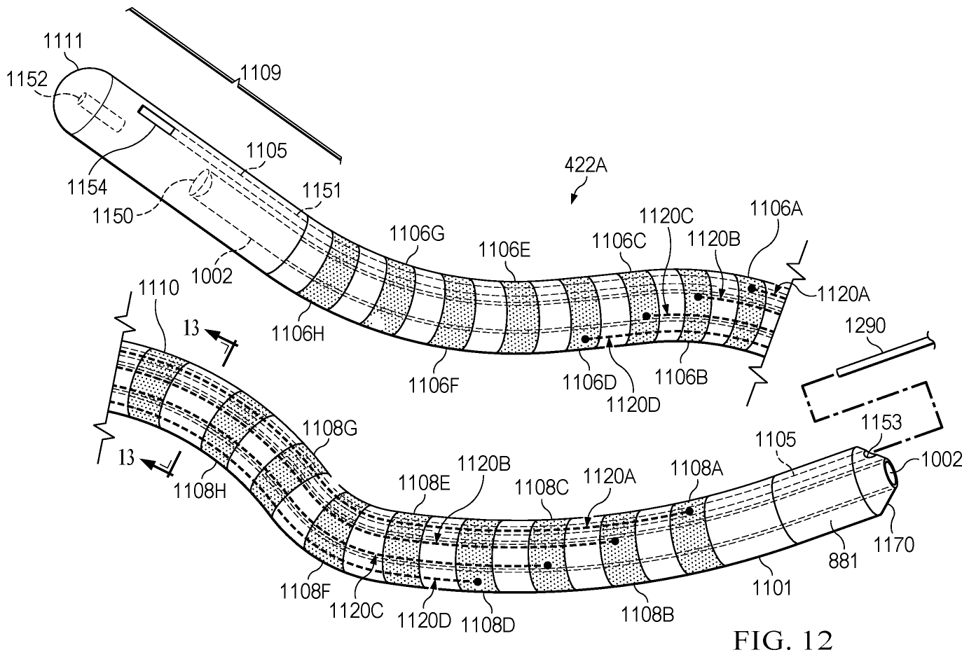

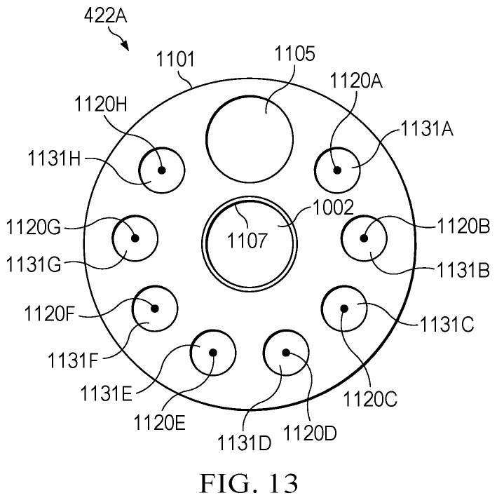

Referring then to FIGS. 12 and 13, lead 422A will be further described. Leads 422A and 422B are identical in structure and function. Only lead 422A will be described here, by way of example.

Lead 422A further comprises lead body 1101. Lead body 1101 is generally a flexible cylindrical extrusion distally terminated by transmission tip 1109 and proximally terminated by collet 881. In a preferred embodiment, the lead body is comprised of a flexible polymer, such as Pellethane 55D or similar biocompatible material. The lead body is preferably a multi-lumen extrusion having embedded and integrally formed components, as will be further described.

Transmission tip 1109 is an optically transparent cylinder fused to the distal terminus of the lead body. In a preferred embodiment, the transmission tip is a suitable optically transparent material such as a thermoplastic polyurethane. Transmission tip 1109 is terminated by semi-spherical cap 1111. In a preferred embodiment, semi-spherical cap 1111 and transmission tip 1109 are integrally formed. Transmission tip 1109 further includes embedded radiopaque marker 1152. Radiopaque marker 1152 is preferably titanium cylinder axially embedded adjacent spherical cap 1111.

Fiber 1002 is positioned along the central optical axis of lead 422A and extends from collet 881 to concave lexicon 1150. The transmission tip is fused to fiber 1002. Fiber 1002 includes concave lexicon 1150 at its distal end. In a preferred embodiment, concave lexicon 1150 includes internally reflective coating such as titanium dioxide. Transmission tip 1109 further includes stylet channel terminus 1151. In a preferred embodiment, stylet channel terminus 1151 is a cylindrical opening. Stylet stop 1154 is positioned at the distal end of stylet channel terminus 1151. Stylet stop 1154 is preferably a titanium cylinder.

Stylet channel 1105 is coaxial with and extends from stylet channel terminus 1151 to stylet channel opening 1153, in collet 881. The stylet channel is a cylindrical cavity which serves the purpose of housing guide stylet 1290 for use during placement of the lead during surgery. In preferred embodiment, stylet channel 1105 is lined with a polytetrafluoroethylene (PTFE) lining 1107, which extends the length of the lead body. The low surface friction afforded by the lining facilitates insertion of the stylet during surgery. In use, the leads are first positioned in holes 525A and 525B. The stylets extend from the leads through open ports 550A and 550B so that the surgeon may adjust placement of the leads while the stimulation signal is active.

Lead body 1101 further supports metallic anchor 1110 positioned at its proximal end. The metallic anchor is generally cylindrical and is permanently affixed to the exterior of the lead body.

Adjacent metallic anchor 1110, are eight cylindrical proximal metallic contacts, 1108A, 1108B, 1108C, 1108D, 1108E, 1108F, 1108G and 1108H are fixed to the exterior of the lead body at even axial distances and are each positioned to electrically contact one pair of the spring contacts in the header assembly.

Likewise, eight cylindrical metallic electrodes 1106A, 1106B, 1106C, 1106D, 1106E, 1106F, 1106G and 1106H are fixed to the distal end of the lead body. The metallic electrodes are each permanently fixed to the exterior surface of the lead body, at equal axial distances.

The lead body further comprises eight radially oriented lumens, 1131A, 1131B, 1131C, 1131D, 1131E, 1131F, 1131G and 1131H. Conductors 1120A, 1120B, 1120C, 1120D, 1120E, 1120F, 1120G and 1120H are integrally formed in the lumens and extend from their respective proximal contacts to their respective distal electrodes. In a preferred embodiment, the conductors are comprised of MP35N, or another conductive material similarly resistant to corrosion. Each of the conductors to connect exactly one proximal contact with exactly one paired metallic electrode.

In a preferred embodiment, fiber 1002 and the conductors are integrally formed into the lead body during manufacture.

In a preferred embodiment, collet 881 is formed from a suitable ceramic or sapphire material. Collet 881 includes frustoconical surface 1170 at its proximal end. The frustoconical surface mates with an identical frustoconical receiver surface 1050 in header body 800 and aids in positioning fiber 1002 against optical interface 1012, and in radially compressing the fiber to aid in optical alignment with the parabolic redirector.

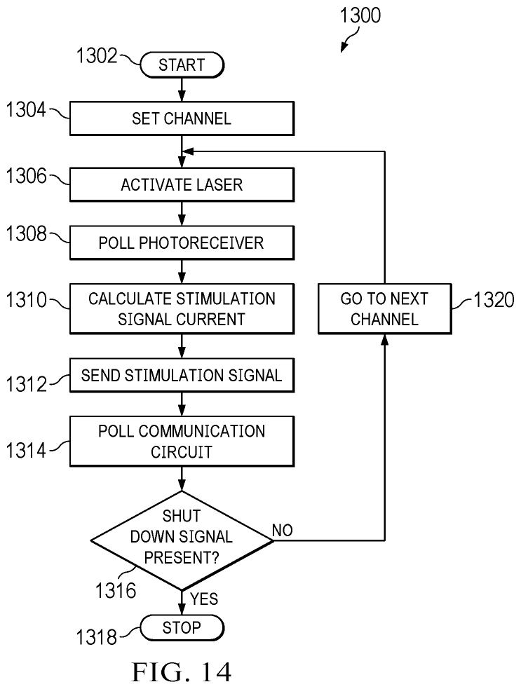

Referring to FIG. 14, a method of EPG operation 1300 will be further described. In preferred embodiment, the method is carried out by programming instructions, which are resident in onboard memory of main processor 407.

At step 1302, the method begins.

At step 1304, the main processor sets an initial channel for operation. In a preferred embodiment, an initial channel includes one of leads 422A or 422B. Each of the eight electrodes on the lead selected may be individually addressed by the main processor with a different current level for the stimulation signal.

At step 1306, main processor 407 preferably activates the IR crystal of the VCSEL for the specified channel. The VCSEL sends a light pulse to the base of the prism. The angled surface of the prism turns the light about 90 degrees from the vertical to the horizontal and reflects it toward the interface surface adjacent the parabolic redirector and toward the parabolic surface. The parabolic surface turns the light horizontally about 90 degrees and collects and focuses it through the integral lens to the optical axis of the optical fiber for the chosen lead, where it traverses the lead and exits from the concave lexicon through the transmission tip. The transmitted ray then is incident on the spinal cord, hemoglobin and other surrounding tissues, where it is reflected and received by the fiber at the concave lexicon as a received ray. The received ray is transmitted down the fiber returning to the parabolic redirector where it is turned about 90 degrees horizontally and toward the prism, which turns the light about 90 degrees from the horizontal downward vertically, and focuses it on the photoreceiver of the die stack.

At step 1308, the main processor polls the photoreceiver adjacent the chosen lead terminus for a current signal.

At step 1310, the main processor calculates a stimulation signal based on the signal from the photoreceiver. The stimulation signal is preferably generated according to a table, accurately disclosed in U.S. Pat. No. 9,550,063 to Wolf II, incorporated herein by reference. Of course, other stimulation routines may be used.

At step 1312, the main processor sends the stimulation signal to the spring contacts on the lead for the chosen channel. The stimulation signal is transmitted to the electrodes, which creates an electric field adjacent the target neurons at the spinal cord.

At step 1314, the main processor polls the communication circuit for a shutdown signal.

At step 1316, the main processor determines whether or not a shutdown signal is present. If not, the main processor moves to step 1320. If so, the main processor moves to step 1318.

At step 1320, the main processor advances to the next lead channel and returns to step 1306.

At step 1318, the main processor shuts down the routine and returns to a holding state.

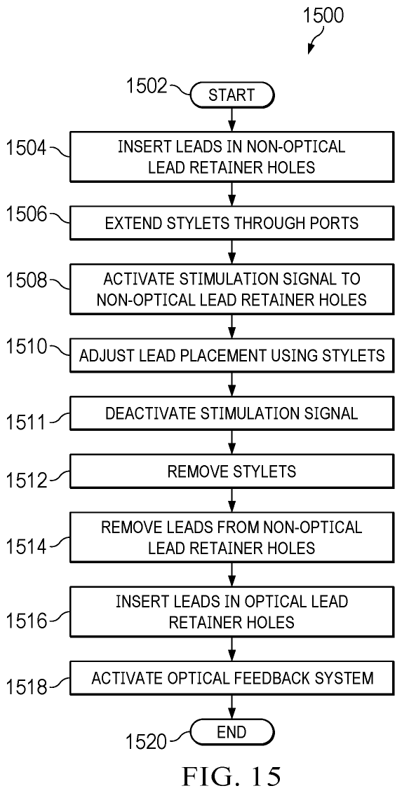

Referring to FIG. 15, preferred method of use of the EPG 1500 will be further described.

During lead placement, positioning of the leads to effectively transmit the stimulation signal to the spine is difficult and requires great surgical acuity. Complicating the problem is that the stimulation signal must be present during lead placement in order to elicit patient feedback as to stimulation efficacy. Further complicating the problem is that use of the stylet in the lead interferes with placement of the lead adjacent the optical redirectors. To avoid these problems, leads 422A and 422B are first positioned in non-optical lead retainer holes 525A and 525B. Then when positioning is complete, they are moved to optical lead retainer holes 523A and 523B, according to the following method.

At step 1502, the method begins.

At step 1504, leads are inserted in the non-optical lead retainer holes.

At step 1506, the stylets for the leads are positioned through the access ports in the header assembly.

At step 1508, the signal generator is activated to generate a stimulation signal to the spring contacts in the non-optical lead retainer holes.

At step 1510, lead placement is adjusted using the stylets.

At step 1511, upon optimal lead placement, the signal generator is deactivated to stop the stimulation signal.

At step 1512, the stylets are removed from the leads.

At step 1514, leads are removed from the non-optical lead retainer holes.

At step 1516, leads are inserted in the optical lead retainer holes and positioned so as to abut the collets and the leads against the optical redirectors.

At step 1518, the optical feedback system of the EPG is activated, thereby sending a controlled stimulation signal to the electrodes.

At step 1520, the method concludes.

1. An external pulse generator system comprising:
a case;
a lead retainer hole, positioned in the case, having a first optical axis;
a parabolic redirector, having a first interface surface, perpendicular to a second interface surface, connected by a parabolic surface, focused on the first optical axis;
a right angle prism, having a third interface surface perpendicular to a fourth interface surface connected by an angled surface, adjacent the parabolic redirector;
the second interface surface immediately adjacent the third interface surface;
a laser, directed toward the fourth interface surface;
a photoreceiver, surrounding the laser, positioned parallel to the fourth interface surface; and
a processor circuit, having a memory, operatively connected to the laser and the photoreceiver.

2. The external pulse generator system of claim 1, wherein the parabolic redirector incorporates a collimating lens, adjacent the first interface surface, focused on the first optical axis.

3. The external pulse generator system of claim 1, wherein the laser is fixed on the photoreceiver by a transmission window.

4. The external pulse generator system of claim 3, wherein the laser is electrically connected to the processor circuit by a metallic trace on the transmission window.

5. The external pulse generator system of claim 1, wherein the parabolic surface has a first reflective coating.

6. The external pulse generator system of claim 5, wherein the angled surface has a second reflective coating.

7. The external pulse generator system of claim 1, further comprising:
a percutaneous lead, fixed in the lead retainer hole, having a second optical axis coaxial with the first optical axis;
an optical fiber, axially positioned along the second optical axis, integrally formed in the percutaneous lead;
a set of electrical contacts, proximally fixed on an exterior surface of the percutaneous lead, electrically connected to the processor circuit; and
a set of stimulation electrodes, distally fixed on the exterior surface of the percutaneous lead, electrically connected to the set of electrical contacts, by a set of flexible conductors, integrally formed in the percutaneous lead.

8. The external pulse generator system of claim 7, wherein the percutaneous lead further comprises:
a stylet lumen, disposed adjacent and parallel to the optical fiber.

9. The external pulse generator system of claim 8, wherein the percutaneous lead further comprises:
a transparent optical transmission tip optically fused with the optical fiber.

10. The external pulse generator system of claim 9, further comprising:
a set of instructions, resident in the memory, that when executed cause the external pulse generator system to:
generate a transmit ray from the laser;
send the transmit ray, through the right angle prism and the parabolic redirector to the first optical axis;
receive a reflected ray from the optical fiber, through the parabolic redirector and a prism, incident on the photoreceiver;
generate a variation variable based on the reflected ray;
generate an electrical stimulation signal, modulated by the variation variable; and
send the electrical stimulation signal to the set of electrical contacts to create a modulated electrical field at the set of stimulation electrodes.

11. An external pulse generator system comprising:
a case;
an electro-optical lead, positioned in the case, having an optical axis;
an optical folding assembly, for turning the optical axis about 90 degrees horizontally to a horizontal axis and about 90 degrees vertically to a vertical axis; and
a die stack assembly, for sending transmit light pulses to the vertical axis, and for collecting receive light pulses, from the vertical axis.

12. The external pulse generator system of claim 11, further comprising:
a processor circuit, operatively connected to the die stack assembly, for activating the die stack assembly to send the transmit light pulses and for interpreting the receive light pulses, and for modulating a stimulation signal based on the interpretation.

13. The external pulse generator system of claim 11, wherein the electro-optical lead further comprises:
a centrally disposed optical fiber, integrally formed in the electro-optical lead, coaxial with the optical axis;
a set of metallic contacts, fixed on an exterior surface of the electro-optical lead;
a set of metallic electrodes, formed on the exterior surface of the electro-optical lead opposite the set of metallic contacts;
a set of wires, integrally formed in the electro-optical lead, connecting the set of metallic contacts and the set of metallic electrodes; and
a longitudinal stylet lumen, adjacent the centrally disposed optical fiber.

14. The external pulse generator system of claim 13, wherein the longitudinal stylet lumen is proximally terminated at a rounded portal, positioned in a frustroconical surface of a rigid collet and distally terminated by a rigid stylet stop.

15. The external pulse generator system of claim 11, wherein the optical folding assembly further comprises:
a parabolic reflective surface, for turning the optical axis about 90 degrees horizontally and for focusing a light signal on the optical axis; and
an angled flat reflective surface, for turning the optical axis about 90 degrees vertically, and for directing the light signal to the die stack assembly.

16. The external pulse generator system of claim 15, wherein the parabolic reflective surface and the angled flat reflective surface are integrally formed.

17. The external pulse generator system of claim 15, further comprising:
a collimating lens, adjacent the parabolic reflective surface, collinear with the optical axis.

18. The external pulse generator system of claim 15, wherein:
the parabolic reflective surface has a first exterior reflective coating; and
the angled flat reflective surface has a second exterior reflective coating.

19. The external pulse generator system of claim 11, wherein the die stack assembly further comprises:
a laser, directed along the vertical axis; and
a photodiode, positioned around the laser, oriented perpendicularly to the vertical axis.

20. The external pulse generator system of claim 11, wherein the optical folding assembly further comprises:
a right hand parabolic redirector diametrically opposed to a left hand parabolic redirector.

21. An external pulse generator system comprising:
a case;
a first electro-optical lead, having a first optical axis, removably fixed in the case;
a second electro-optical lead, having a second optical axis, removably fixed in the case;
a left hand optical redirector for turning the first optical axis counterclockwise horizontally and vertically downward;
a right hand optical redirector, diametrically opposed to the left hand optical redirector, for turning the second optical axis clockwise horizontally and vertically downward;
a first laser, directed toward the left hand optical redirector;
a second laser, directed toward the right hand optical redirector;
a first photoreceiver, surrounding and perpendicular to the first laser;
a second photoreceiver, surrounding and perpendicular to the second laser; and
a processor circuit, operatively connected to the first laser and the second laser, the first photoreceiver and the second photoreceiver.

22. The external pulse generator system of claim 21, wherein the processor circuit is programmed to:
send a first laser pulse, on the first electro-optical lead, from the first laser; and
send a second laser pulse, on the second electro-optical lead, from the second laser.

23. The external pulse generator system of claim 22, wherein the processor circuit is programmed to:
receive a third laser pulse at the first photoreceiver; and
receive a fourth laser pulse at the second photoreceiver.

24. The external pulse generator system of claim 23, wherein the processor circuit is further programmed to:
derive a first compensation value from the third laser pulse;
derive a second compensation value from the fourth laser pulse;
modulate a first stimulation signal based on the first compensation value;
modulate a second stimulation signal based on the second compensation value;
send the first stimulation signal on the first electro-optical lead; and
send the second stimulation signal on the second electro-optical lead.

25. A method of use of an external pulse generator comprising:
providing a non-optical lead retainer hole in the external pulse generator;
providing an optical lead retainer hole in the external pulse generator;
inserting an electro-optical lead, including a stylet, in the non-optical lead retainer hole;
activating a stimulation signal to the electro-optical lead;
adjusting placement of the electro-optical lead using the stylet;
deactivating the stimulation signal;
removing the electro-optical lead from the non-optical lead retainer hole;
inserting the electro-optical lead in the optical lead retainer hole; and
activating an optical feedback system in the external pulse generator.

26. The method of claim 25 further comprises:
adjusting the stimulation signal according to an optical reflection from the optical feedback system.
